(19) United States
(12) Patent Application Publication
(10) Pub. No.: US 2009/0170175 A1
(43) Pub. Date: Jul. 2, 2009

(54) PROCEDURE AND ARRANGEMENT FOR TOASTING CORN AND THE PRODUCT OBTAINED TO BE APPLIED IN THE PREPARATION OF MASH FOR ETHYL FERMENTATION

(76) Inventor: Jose Antonio Lopez Hernandez, Cordoba (AR)

Correspondence Address: BACHMAN & LAPOINTE, P.C., 900 CHAPEL STREET, SUITE 1201, NEW HAVEN, CT, 06510, US

(21) Appl. No.: 12/167,674

(22) Filed: Jul. 3, 2008

(30) Foreign Application Priority Data
Dec. 28, 2007 (AR) P070105985

(51) Int. Cl. C12P 7/06; B01J 19/18

(52) U.S. Cl. 435/161; 422/225

(57) ABSTRACT

A procedure to turn the starch contained in corn grains to soluble forms includes seven steps, starting a first step in which the corn that comes from a hopper is placed into a rotary and continuous oven for its toasting, characterized because it also links a second step in which the corn is toasted by injection of combustion gases to a temperature between 225 and 250° C. during 80 and 120 seconds to obtain a 48% of the popped grains and a 28% of semi-popped grains; then the third step consists of milling the toasted product; the fourth step consists that the milled product is placed into a blade mixer, water is added at a temperature of between 20 and 25° C. and it is mixed up to obtain the mash; in the fifth step, the mash is put into saccharification tanks, adding a saccharifying agent and is mixed, a sixth step consists of transferring the mash to a fermentation vessel and the seventh step is the distillation of the ethanol produced.

### BACKGROUND OF THE INVENTION

(1) Field of the Invention

The invention relates to a procedure and arrangement for toasting corn and the product obtained to be applied in the preparation of mash for ethyl fermentation.

(2) Prior Art

Exploitation of non-renewable resources to obtain fuel seems to have a future end that the prospecting and exploration seem to confirm daily.

It is highly probable that the internal combustion engine, in spite of the serious effects produced by the resulting gases in the atmosphere, will still have many years of use. In fact, in spite of being an inefficient and pollutant machine, the internal combustion engine is distributed in great number all over the world. According to the news, in the next five years, the markets of China and India have plans to increase their car fleet in a percentage that goes from 8% to 20% respectively. This implies that between these two countries alone, the worldwide car fleet will increase at about 1,500,000 vehicles per year. Considering the issue from this point of view, the truth is that if the internal combustion engine is not replaced, we should obtain a greater efficiency in the use of fuel that is used, and also try to replace fossil fuel, non-renewable, with renewable biofuel.

Among the measures prone to replace fossil fuel with biofuel, the Federal Republic of Brazil is carrying out a project with the so-called alcopetrol, that is an alcohol obtained from the fermentation of fruits with a high content of sugar. On the other hand, the United States of America has decided that 5% of fossil fuel in use should be replaced with ethanol in a project in which the percentage of this alcohol will go increasing slowly up to the 20%. Considering these last figures, it is estimated that the replacement of the 20% of the fuel will require 100,000 million of liters of ethanol. To give an idea of its importance, this is five times greater than the total current production of Brazil.

### SUMMARY OF THE INVENTION

In accordance with the present invention, a procedure and an arrangement have been designed that make an important economy possible in terms of the cost both the necessary installations and the thermal energy use. The saving in the installations is produced considering that for the case being developed, boilers to produce steam and a toasting oven are used. Installations are complemented with a mill, mixer, saccharification tanks, fermentation vessels and fluid containers necessary for the procedure described below. With the procedure that is being revealed, amylodextrins are obtained and are soluble in water at low temperature making unnecessary the use of energy to increase the temperature of the solvent. On the other hand, the very fast toasting process, in relation to the continuous cooking method, also enables the saving of energy in this stage.

As described herein, a procedure to turn starch contained in corn grains to soluble forms includes seven steps, starting with a first step in which the corn that comes from a hopper is placed into a rotary and continuous oven for its toasting, a second step in which the corn is toasted by injection of combustion gases to a temperature between 225 and 250° C. during 80 and 120 seconds to obtain 48% of popped grains and a 28% of semi-popped grains; a third step consisting of milling the toasted product; a fourth step consisting of the milled product being placed into a blade mixer, adding water at a temperature of between 20 and 25° C. and mixing it up to obtain a mash; in a fifth step, the mash is put into saccharification tanks, adding a saccharifying agent and mixing the two; a sixth step consisting of transferring the mash to a fermentation vessel; and a seventh step which is the distillation of the ethanol produced.

In order to get a better understanding of this invention to be put into practice with ease, the following provides a detailed description of the way to carry out the invention, making reference in the description to the attached drawings, the whole with character of purely demonstrative example but not restrictive to the invention. Its components will be able to be selected among several equivalents without leaving aside the principles of the invention.

### BRIEF DESCRIPTION OF THE DRAWING(S)

In FIG. 1, the different components that enable one to carry out the procedure that is being disclosed are outlined.

FIG. 2 is a graph showing liters vs. time.

### DETAILED DESCRIPTION OF THE PREFERRED EMBODIMENT(S)

In FIG. 1, the same reference characters indicate equal or corresponding parts, being number 1 a hopper; number 2 an oven; number 3 an exit for gases; number 4 a mill; number 5 a mixer; number 6 a tank; number 7 a saccharification tank; number 8 a container; number 9 a fermentation vessel and number 10 an exit pipe.

The invention consists of a series of steps that enable the starch contained in corn grains to go to soluble forms. The first step is to place the corn coming from a hopper in an oven; the second step consists of toasting the corn; the third step is to mill the toasted product; the fourth step is to place the toasted and milled product into a mixer, add water and mix; the fifth step consists of entering the resulting mash in saccharification tanks, add a saccharifying agent and mix, and the sixth step is to transfer it to a fermentation vessel for its distillation.

In order to obtain the procedure described, an arrangement is used that consists of a hopper (1), an oven (2), a mill (4), a mixer (5), a tank (6), saccharification tanks (7), a container (8) and a fermentation vessel (9).

Once the step sequence is established in order to explain the nature of the invention, it is then complemented with their functional and operating relation and the result they provide. In order to obtain a procedure that enables to go from the starch contained in the corn grains to soluble forms, it has been determined that the procedure should be the one of toasting. It is necessary to clarify that the term toasting, as used herein, used herein, means to heat the grain abruptly so that it pops or cracks open. When heating the grain, the water contained as humidity starts evaporating, and comes out through the pores that are in the coating. However, if the heating was produced abruptly, the pores would not be enough to remove the steam produced by the humidity of the grain, since it is estimated that it could have approximately 40 times the volume of the water contained. This abrupt steam release would be translated into a rise in the internal pressure that, acting over the starch, would gel it until the resistance of the grain is overcome by the pressure, producing its pop.

Several toasting tests have been carried out in which it has been tried to determine the most economical toasting temperature; that is to say, to find the point of equilibrium between the energy used and the product obtained. In those tests, the grain never touches the walls of the container so the heat of the flame applied on the base of the container comes to the grain only by radiation. In all the tests, a blackplate container of 2 mm thick and 200 mm edge was used. The inside part of the container was insulated with an asbestos sheet and a wire mesh as a mezzanine in order to withhold 50 grams of corn.

Test 1: The oven temperature was taken to 175° C. and the corn was put inside. A 17% of the grains popped and they took three minutes in doing it. The burning time was of 30 minutes.

Test 2: The oven temperature was taken to 200° C. and the corn was put inside. A 16% of the grains popped and a 52% remained semi-popped. The necessary time to produce the pop of the grains was of two minutes and the burning time was 20 minutes.

Test 3: The oven temperature was taken to 250° C. and the corn was put inside. A 48% of the grains popped and a 28% remained semi-popped. The necessary time to produce the pop of the grains was 1.5 minutes and the burning time was five minutes.

Test 4: The oven temperature was taken to 300° C. and the corn was put inside. A 56% of the grains popped and a 20% remained semi-popped. The necessary time to produce the burst of the grains was 35 seconds and the burning time was three minutes.

From these tests, it is deduced that the grains that did not pop increase to 24% in the Tests 3 and 4 while the increase of 50° C. is translated in an 8% more of popped grains at the expense of equal reduction of semi-popped grains. This gives us approximately the same toasting effect with temperature from 250 to 300° C.

The comparison now goes to the solubility test of the corn starch toasted at the different temperatures of the tests abovementioned in order to determine what the minimum temperature of a constant volume of water is able to solubilize the maximum of a constant weight of toasted corn in a certain time.

In order to get the answer, 5 grams (gr.) of toasted corn with 100 cc of water warmed at different temperatures between 20 and 80° C. were put in an Erlenmeyer. The temperature of the water remained constant for 10 minutes. The content of the Erlenmeyer was placed into a test tube to decant the insoluble parts of the grain together with the insoluble saccharifiable part that would be constituted by the raw starch to proceed then to throw out the upper liquid part.

The steps were repeated until the upper liquid part did not react to iodine. In this opportunity, the decanted part is recovered and hydrolyzed in an autoclave so that glucose is then valorized. The result obtained will be the amount of toasting that cannot be solubilized to the water temperature selected to conduct the experiment.

As the soluble saccharifiable portion gives a shade of violet coloring due to iodine reaction, it should be determined if the reaction is produced due to the presence of soluble starch or amylodextrin, consequently a differential reaction was carried out (see Allem “Commercial Organic Analysis”, Vol I, 527) which determined that the reaction matched the presence of a dextrin mixture which includes amylodextrin and eritrodextrin. This differentiation, for the purpose of the object of invention, becomes purely statistical and that is the reason why individualization and quantification were not conducted.

Experiment 1: In this case, toasted corn was used at 175° C. and 55% of dextrins could be solubilized with water at 20° C. Increasing the temperature of water being used by 10° C. each time, 62.3% of dextrins were solubilized at 30° C., 68.9% at 40° C., 40° C., 76.9% at 50° C., 84.9% at 60° C. and 100% dextrins were finally solubilized at 70° C.

Experiment 2: In this case the material obtained through toasting at 250° C. was used and it was determined that with water at 20° C., 91.5% dextrins are solubilized, 99.1% with water at 30° C., and then 100% with water at 40° C.

As it can be seen in Example 1, it is with water at 70° C. that the whole material is solubilized. This shows that toasting at 175° C. has not obtained the total amount of starch since it is precisely at 70° C. that the temperature of starch solubilization is obtained.

In this order of ideas, being approximately 30° C. the temperature for dextrin solubilization, the conclusion can be that in the material obtained through toasting at 250° C. there are no starch remains.

As we have seen in the tests, the use of temperatures above 225° C. enables one to obtain dextrins and thus the consequent process of saccharification is shortened significantly.

In order to determine the saccharification time required from the toasted material compared to that of the brewed material; a tinned steel fermentation vessel provided with a variable speed blade agitator was used.

In order to keep fermentation temperature stable, the vessel has an automatic regulation system which consists of a thermoelectric couple.

In order to conduct the first saccharification, amylasa from Mucor Delemar fungus was sowed in 3.5 liters of water at 38° C. where 300 gr. of toasted milled product had been previously added.

After ten hours from sowing, the reaction tests to iodine started, and it was checked that after eighteen hours the saccharification was complete.

To conduct saccharifications comparable between product obtained through toasting and product obtained through a continuous cooking method, the use of fungi was left aside since the biological elements can be influenced by many factors over which a full control cannot be exerted.

As a consequence, “Takadiastasa” was used, which is an amylasa obtained through alcohol precipitation of cultures where amylolitic fungi were developed. Takiadastasa was added to the mash prepared with water and aggregate of milled product obtained through toasting and through a continuous cooking method. It was determined that the saccharification time of the mash contained in the product obtained through toasting resulted 25% faster than the mash containing the product obtained through a continuous cooking method.

One of the reasons of this higher velocity brings as a consequence that in the mash containing toasted product, the saccharification phase starts from dextrins while in the mash containing product obtained through a continuous cooling method the saccharification process is started with starch, that is to say, in a previous stage.

With the procedure of this invention, the toasting effect turns the starch to a soluble state such as dextrins result.

The procedure of toasting requires two minutes at 250° C. while the time required by saccharification of the same weight of carbon hydrates by the action of same amounts of amylasa is lower in a 25% in the mash with toasted product (18 hours) compared to the brewed product (24 hours).

The amount of heat consumed in toasting an amount of corn enough to produce 1 liter of alcohol through fermentation corresponds to the formula:

Q: 2.8×0.32(250−25)=201.6 Cal.

Considering that the toasting procedure is carried out in a continuous rotary oven, we should take into account that the drop in temperature between the access end and the output end of the oven is 50° C. and knowing that the amount of heat that combustion gases have at 250° C. is 134 Cal. (calories) and 118 Cal. at 200° C., it has been concluded that the difference between both is the amount of heat given to the corn for each kilogram of combustion gas, that is to say 16 Cal.

We know that for each kilogram of wood one obtains about 3500 Cal. in a fireplace, which is equivalent to 27 kg. of combustion gases to 250° C. which, at this temperature, represent a volume of 67.5 m³.

In order to carry out the complete combustion of 1 kg. of wood, about 10 m³ of air are required, that is why in the fireplace a secondary air volume of 57.5 m³ must be introduced.

Being 201.6 Cal. the amount necessary to toast 2.8 kg. of corn and obtain 1 liter of alcohol and considering that because of temperature gradients between the ends of the oven, only 16 Cal. are given to the corn, it will be necessary to use 12.6 kg. of combustion gas per liter of alcohol obtained.

From this, it can be inferred that 118 Cal. are expelled through gas exit and that is the reason why it is appropriate to use this hot air to re-feed the boilers that will give the 250° C. required for the most effective toasting.

The toasted product is taken to the mill to obtain its disgregation. Due to the toasting process, the product becomes soft to the point that with finger pressure can be disgregated, reason by which the energy necessary to carry out the milling is pretty lower than the one necessary to mill the raw grain.

This is a consequence of starch granule destruction that, previous to toasting are connected by a corneal clogging that constitutes the endosperm.

The mixture of the milled toasted product with cold water makes up what is called mash, which, as a consequence of the heat it was brought inside the oven, it is sterilized. Consequently, it is appropriate that the water to be used must also be sterilized in order not to contaminate the brew and have the mash ready for saccharification.

We know that the toasted product leaves the oven at a temperature of 200° C., consequently we obtain 60 Cal. per kg. of toasted material, which will be transmitted to the water.

We also know that approximately 3.5 liters of water are needed per kg. of toasted product, reason by which, with water added at 22° C. the calories given by the toasted product will enable to rise its temperature in 38° C. to take it to 60° C.

By virtue of what has been said before, the procedure that is revealed consists of a first step in which the corn that comes from a hopper is placed into a rotary and continuous oven for its toasting.

It also consists of a second step in which the corn is toasted taking it to a temperature of between 225 and 250° C. during a time between 80 and 120 seconds to obtain a 48% of popped grains and a 28% of semi-popped grains.

In a third step, the toasted product is milled; in a fourth step it is placed in a blade mixer, adding water at a temperature of about 20 and 25° C. to obtain the mash.

In a fifth step, the mash is entered into saccharification tanks, a saccharifying agent is added and then it is mixed. In this step, the saccharifying agent is the amylase obtained from the Aspergillus Niger or barley malt, that contains the same enzyme.

In a sixth step, the mash is transferred to a fermentation vessel from where in a seventh step the ethanol obtained is sent to its distillation.

Therefore, the arrangement required to carry out the procedure described above includes a hopper (1) where the grain to be toasted is placed. This hopper (1) allows the passage of the grain to an oven (2) that enables to reach a continuous toasting temperature of between the 250 and 300° C. Preferably, this oven (2) will be the rotary and continuous type and the toasting of the kernels will be by means of the injection of direct combustion gases.

The oven (2) has a gas exit (3) that, since its temperature will be less in some 50° C. with regard to the entrance gas, is appropriate to be used as air supply in the boiler room. The oven (2) also counts with an exit of the toasted product that is linked to a mill (4) where the grain is milled to be sent then to the mixer (5) where cold water is added. The water comes from the tank (6) and is mixed with the toasted and milled grain thanks to the blades added to the mixer.

The mixture of toasted and milled grain with water is sent to the saccharification tanks (7) in which a solution of funginus amylase or of malt is added from a container (8).

The obtained saccharified mash is sent to the fermentation vessel (9) where once the fermentation is produced, this is sent through the exit pipe (10) to the distillation tanks.

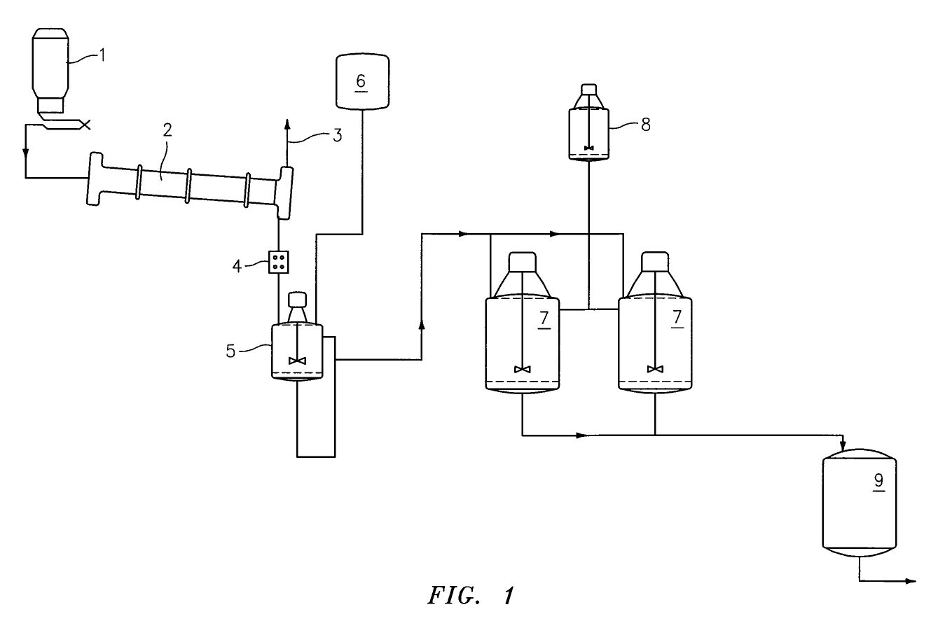

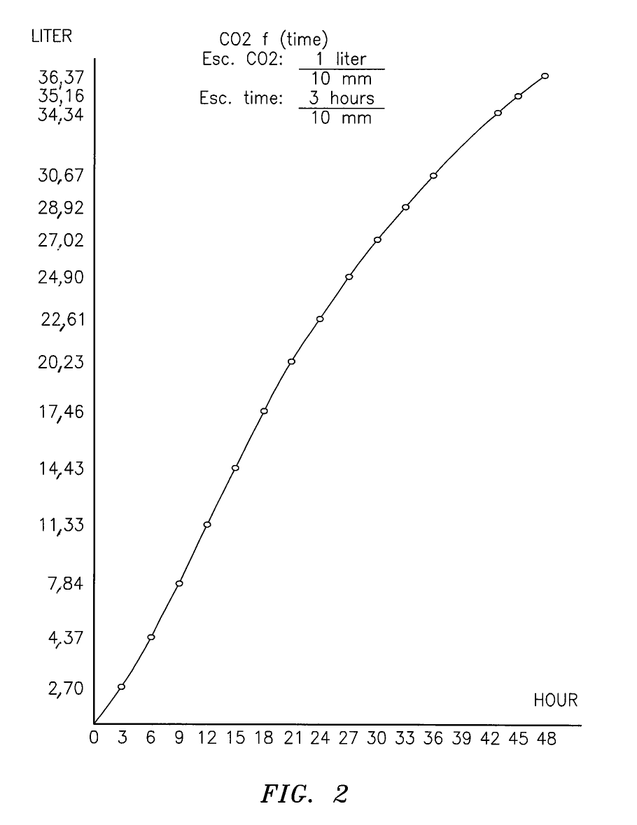

The saccharified mash was subjected to a fermentation test that consisted in lowering the temperature first to 27° C. to then sow a lab culture of Saccharomyces Cereviseae. After five hours, the ventilation and the mixing was cut, leaving to ferment for 48 hours in which readings of the carbon dioxide emission were done every half an hour. These values have been added to the graph of FIG. 2 of which not only the normal development of the fermentation but also the property of the yeast of being developed normally in mash of toasting is deduced. Once the fermentation was finished, the distillation process started in the same fermentation vessel (9) sending water to the jacket and collecting the alcoholic steam in the loading nozzle to condensate them in a Liebig's cooler.

Then a distillation of exhaustion was made, taking different portions of distillate and taking them to 15° C. to take density and express the alcoholic content in alcohol of 100° C. G.L. in weight.

The density measurements obtained have been written down in the attached sheet indicated as Appendix A.

With regard to the fermentation performance, it was determined that the toasting used had a content in dextrin equivalent to the 72% expressed in glucose, percentage that decreased from the original 79.2% at the oven exit as a consequence of the water added between the day of going out from the oven and the day of its use.

According to the following ratio:

$$\frac{2 C 2 H 5 OH}{C 6 H 1206} = \frac{92}{180} = 0.5112$$

We can state that a part of glucose enables to obtain 0.5112 parts of alcohol.

Consequently, if 300 gr. of toasting have been used with 72% of glucose, it determines that the amount of glucose used is 216 gr., this value arises from multiplying the grams of toasting by the percentage of glucose contained and dividing it by one hundred.

Knowing that each part of glucose enables to obtain 0.5112 parts of alcohol, the real 216 gr. of glucose enables, at least in theory, to obtain 110.4 gr. of alcohol.

As according to the figures written down in Appendix A, the alcohol obtained was 100 gr., we can conclude that the effectiveness of the procedure is of the 90.5%. The yeast used is the same as the one used to prepare both the mash of toasting and and the mash by a continuous cooking method.

According to what was previously exposed, it enables to reach the conclusions as follows:

1. Due to the toasting, the starch contained in the corn goes to soluble forms (dextrins).

2. The dextrins obtained by the toasting procedure revealed herein are saccharifiable.

3. The verifications of alcoholic fermentation practiced in the mash of saccharified toastings give normal values.

4. The total time of toasting at 250° C. is two minutes.

5. The total time of saccharification of a mash of toasting is 25% less than the saccharification time of the same weight of carbohydrates by action of equal quantities of amylase.

6. The amount of heat consumed during the toasting process at 250° C. of enough corn to obtain a liter of alcohol by fermentation is 201.6 Cal. as it is obtained from the formula:

Q=2.8·0.32(250÷25)=201.6

7. Due to the toasting process, the resulting product appears sterilized.

In this way, the constructive possibilities that help to carry out the invention and the way in which the same works has been depicted, and documentation is complemented with the synthesis of the invention contained in the claims that are added hereinafter.

What is claimed is:

1. A procedure for turning starch contained in corn grains into soluble forms, said procedure including a first step where corn coming from a hopper is placed into a rotary and continuous oven for its toasting, a second step where the corn is toasted by direct combustion gases to a temperature of between 225 and 250° C. during a period between 80 and 120 seconds to obtain 48% popped grains and 28% of semi-popped grains; a third step of milling the toasted corn product; a fourth step where the milled corn product is placed into a blade mixer, adding water to a temperature of between 20 and 25° C. and mixing to obtain a mash; a fifth step where the mash is put into saccharification tanks, adding a saccharifying agent and mixing the mash and saccharifying agent a sixth step of transferring the mash to a fermentation vessel and a seventh step of distilling produced ethanol.

2. The procedure according to claim 1, wherein said saccharifying agent adding step comprises adding amylase obtained from Aspergillus Niger or barley malt.

3. An arrangement to turn starch contained in corn grains to soluble forms including a hopper that contains the grains to be toasted that goes to a rotary and continuous oven provided with a gas exit which is connected to steam boilers and with an exit of toasted product towards a mill from which the milling is sent to a receiving mixer which receives cold water that comes from a tank; said mixer being linked at least with a saccharifying tank linked to a container of a funginus amylase to send saccharified mash to a fermentation vessel and, through an exit pipe to distillation tanks.

4. The arrangement according to claim 3, wherein gases exiting through the gas exit feed back to the steam boilers.

5. The arrangement according to claim 3, wherein the mixer has a plurality of blades.
